(19) United States
(12) Patent Application Publication
(10) Pub. No.: US 2013/0157922 A1
(43) Pub. Date: Jun. 20, 2013

(54) WATER-BASED GEL WITH LOW SYNERESIS

(71) Applicant: The Dial Corporation, Scottsdale, AZ (US)

(72) Inventors: Tyler R. Mikkelsen, Scottsdale, AZ (US); Richard F. Theiler, Scottsdale, AZ (US)

(73) Assignee: The Dial Corporation, Scottsdale, AZ (US)

(21) Appl. No.: 13/711,040

(22) Filed: Dec. 11, 2012

Related U.S. Application Data
(60) Provisional application No. 61/577,718, filed on Dec. 20, 2011.

(51) Int. Cl. A61L 9/012

(52) U.S. Cl. CPC A61L 9/012
USPC 512/4; 536/91

(57) ABSTRACT

The present invention is a water-based carrageenan gel composition that exhibits remarkable resistance against syneresis and mottling, made possible by the addition of relatively small amounts of nonionic cellulose derivatives such as methyl hydroxyethyl cellulose.

### FIELD OF INVENTION

The present invention relates to an air freshener and in particular to a water-based gel air freshener with improved phase stability.

### BACKGROUND OF THE INVENTION

Gel air fresheners have existed in the market and in the patent literature for decades. Such products are generally water-based and may comprise such few ingredients as a gelling/gellation agent (also referred to as a “gellant”), a fragrance oil, and water. The gelling agent may comprise a polymeric material such as naturally marine-sourced carrageenan, agar, or alginate, or it may comprise a complex blend of materials including carrageenan, a natural gum from non-marine sources, a cellulosic material, and various mono- and divalent cations, used together in various combinations to ensure rigidity of the solidified gel and its stability over time against syneresis. An exemplary gel air freshener product based on carrageenan natural gelling agent is Renuzit® Adjustables® Cone Air Freshener marketed by Henkel. Preferred gel air fresheners are rigid and “self-supporting.” These products look like solids, although closer inspection reveals they are rigid water gels having an outer surface that is easily penetrated during the procedure used to measure gel strength. Some of the prior art relating to the development of gel-based air fresheners follows.

U.S. Pat. No. 2,691,615 (1954, Turner, et al.) is a very early reference claiming a gel based air freshener. The reference discloses the use of agar-agar, gelatin, pectin, starch, and various gums as potential gelling agents for forming air conditioning gels. The aqueous air treating gel comprised of volatile air treatment compounds, water, and 1 to 4% of an aqueous gelling agent, (preferably agar-agar or calcium alginate), was found to be firm and “substantially devoid of syneresis.”

U.S. Pat. No. 2,927,055 (1960, Lanzet) discloses an air-treating gel comprising water, a volatile air treatment component, and a gelling agent mixture comprising carrageenan, Locust Bean gum, potassium chloride, and sodium carboxymethyl cellulose. The mixture is blended at around 170° F., then poured into molds and cooled. The inventors successfully balanced the amounts of these components to improve the viscosity/handling of the gel in the hot/molten state and to optimize stability, firmness, and appearance of the solidified gel.

U.S. Pat. No. 4,056,612 (1977, Lin) discloses an air freshener gel that utilizes a gelling agent mixture comprising carrageenan (mostly kappa and lambda), Locust Bean gum, and an ammonium salt. The inventive gels exhibited high water gel strengths and syneresis rates of less than 0.3%.

U.S. Pat. No. 4,178,264 (1979, Streit, et al.) discloses an improved air-treating gel composition comprising both carrageenan and a stearate salt used in combination as the gelling agent, wherein the preferred ratio of carrageenan to stearate is from about 0.3:1 to about 5:1. In addition to carrageenan, stearate, water, and volatile actives, a stearate solubility enhancer, such as a solvent or one of a variety of nonionic materials, to increase the solubility of the stearate in the aqueous environment. The preferred components for enhancing the stearate solubility include ethylene glycol, propylene glycol, and ethanol. Most of the Streit example compositions comprise propylene glycol, carrageenan, and sodium stearate combinations for rigid and stable gels.

U.S. Pat. No. 4,318,746 (1982, Claffey, et al) discloses a gel having improved physical stability that comprises the combination of a first polymer that dissolves, disperses or hydrates in hot water, and a second polymer that is insoluble in hot water. Claffey discloses that the first polymer is preferably carrageenan and that the second polymer is preferably a cellulose derivative that exhibits reversible thermal gelation properties in water. Such cellulose polymers include methyl cellulose, hydroxypropyl cellulose, hydroxypropyl methylcellulose, and hydroxybutyl methylcellulose.

U.S. Pat. No. 4,666,671 (1987, Purzycki, et al.) discloses fragranced gel blocks useful for deodorizing urinals and toilet bowls. These gel blocks comprise a gelling agent selected from fatty acid salts, sodium alginate, carboxymethyl cellulose, carrageenan, hydroxypropyl cellulose, starches, and gums, although the most preferred gelling agent disclosed is sodium stearate used alone. Solvents including lower alkyl alcohols, diols, and glycol ethers are optionally added to adjust the final melting temperature range of the gel block.

U.S. Pat. No. 5,643,866 (1997, Ansari, et al.) discloses an air-treating gel comprising dibenzylidene sorbitol acetal (DBSA) in combination with a glycol component as the aqueous gelling agent mixture. Such air freshener gels comprising fragrance, water, DBSA and glycol are shaped solid gel products that can withstand temperatures up to 50°-60° C. without melting.

U.S. Pat. No. 5,698,188 (1997, Evans) discloses a gel air fragrancing composition comprising carrageenan in accordance with Lanzet '055. The preferred compositions of Evans comprise 1-20% fragrance, 2-10% carrageenan constituent, and optional preservative and coloring agents, with the balance being water. The carrageenan constituents include commercial thickeners based on carrageenan that are likely to also include proprietary amounts of other materials such as Locust Bean gum, cellulose materials and calcium and/or potassium salts.

U.S. Pat. No. 5,741,482 (1998, Modi) discloses an air treatment gel composition comprising a seaweed gum, a guar or guar derivative, and an additional non-sulfonated water-soluble polymer selected from a group of numerous cellulose polymers.

Lastly, U.S. Patent Application Publication 2008/0317683 (2008, Trudso) discloses carrageenan compositions and products containing these compositions. The disclosure is directed to methods for extracting and producing carrageenans having a mixture of counter-ions (sodium, potassium, calcium and magnesium), wherein the carrageenan composition has a gelling temperature of between 7° C. and 30° C. An air freshener gel is disclosed that comprises the carrageenan composition having the optimized mixture of cations.

In spite of decades of research in the field of water-based gels, the need still remains for new combinations of ingredients that will improve the stability of a water-based gel without adding unnecessary costs.

### SUMMARY OF THE INVENTION

In a preferred embodiment of the present invention, it has been surprising discovered that the addition of small amounts of a nonionic cellulose derivative to an aqueous carrageenan-based air freshening gel will improve stability.

In another preferred embodiment of the present invention, small amounts of methyl hydroxyethyl cellulose, with our without small amounts of hydroxyethyl cellulose, dramatically improves the stability of a carrageenan-based gel.

In another preferred embodiment of the present invention, specific use of the unique cellulose derivative methyl hydroxyethyl cellulose, a cellulosic that exhibits inverse solubility, dramatically improves the freeze-thaw stability of a water-based gel comprising κ-carrageenan from Kappaphycus alverezii “cottonii.”

### DETAILED DESCRIPTION OF THE INVENTION

The following description is of exemplary embodiments only and is not intended to limit the scope, applicability or configuration of the invention in any way. Rather, the following description provides a convenient illustration for implementing exemplary embodiments of the invention. Various changes may be made in the function, size, and arrangement of the elements described without departing from the scope of the invention as set forth in the appended claims. Changes in shape and size of the overall air freshener do not depart from the intended scope of the invention.

That being said, the compositions of the present invention minimally comprise water, fragrance, a carrageenan gelling agent that is preferably κ-carrageenan from Kappaphycus alverezii “cottonii”, and a nonionic cellulose derivative. In a preferred embodiment, the nonionic cellulose derivative comprises alkyl alkoxylated cellulose. In the more preferred embodiment, the cellulose derivative is methyl hydroxyethyl cellulose (MHEC). In order to optimize the physical appearance and stability of the gel air freshener, e.g. to reduce or eliminate syneresis and/or improve appearance after freeze-thaw cycling, the air freshener gel in accordance with the present invention may optionally include other forms of carrageenan (e.g. iota-), gums (e.g. Locust Bean, guar, Cassia, xanthan, etc.), synthetic polyacrylate or polyacrylamide polymers, and/or various ionic agents (e.g. various inorganic or organic salts of Na⁺, K⁺, Ca²⁺, Mg²⁺, and the like).

Carrageenan

Carrageenans are found in abundance in seaweed. These substances are linear sulfated polysaccharides, and more specifically galactans comprising alternating copolymers of α(1→3)-D-galactose and β(1-4→3,6-anhydro-D-galacose units differing in the amounts of sulfate ester groups. A number of structurally different carrageenans are known and have been labeled with Greek letters beta, kappa, iota, mu, nu, lambda, theta, and xi for identification. These galactans differ by the percent and location of the ester sulfate groups and by the percent of 3,6-anhydro bonds, structural differences that give rise to differences in their physical properties. Out of these eight natural carrageenans, kappa, iota, and lambda are the most recognizable for industrial use, with ioda and kappa carrageenan the most important for forming rigid gels. Since the natural carrageenans have sulfate groups (i.e. negative charges throughout the polysaccharide molecule), there are cations associated with carrageenan that can include calcium, sodium, magnesium, and/or potassium ions. The relative amounts of these cations may be altered by the supplier during purification, e.g. by using ion exchange processes, or by the formulator during the manufacturing of the gel, (e.g. by adding other salts when the gel mixture is in the heated, molten aqueous state). Kappa carrageenan (25% ester sulfate; 34% 3,6-AG) is the most cation sensitive carrageenan. It is soluble in hot water and forms a strongly rigid and self-supporting gel in the presence of potassium ions, yet forms brittle gels in the presence of calcium ions. Indeed, it is known that the highest gel strength can be obtained when a gel incorporates kappa carrageenan and potassium chloride (KCl) wherein the KCl is used at about 1.6 wt. % and the kappa carrageenan at 1.0 wt. %, (i.e. a 1.6 to 1 weight ratio of KCl to carrageenan). Iota carrageenan (32% ester sulfate; 30% 3,6-AG) forms elastic gels in the presence of calcium ions. Lambda carrageenan (35% ester sulfate; almost no 3,6-AG), on the other hand, is the least cation sensitive carrageenan and is non-gelling. Therefore, for the purpose of forming air freshener gels that are rigid and self-supporting, kappa and iota carrageenan are more preferred. However, the precise composition of commercially available carrageenans that find use as gelling agents may not be known with any certainty, and may in fact be of proprietary composition. Various suppliers manipulate the source of the carrageenan by farming specific marine species and they customize the purification processes (alkali treatment, ion exchange, etc.) to produce desired mixtures of carrageenans or pure single carrageenan species such as kappa carrageenan.

With the complexities of natural carrageenan in mind, and being aware of the ability of the suppliers to alter their marine sources and purification methods, the general term “carrageenan gelling agent,” an essential ingredient to the stable gel air freshener in accordance with the present invention, refers herein to a gelling agent comprised of any carrageenan (i.e. beta, kappa, iota, mu, nu, lambda, theta, and/or xi) in any conceivable proportion. If unspecified, the carrageenan gelling agent for use herein may comprise very pure kappa carrageenan or it may comprise a mixture of only kappa and iota carrageenans. Generally speaking, if a commercially available carrageenan is marketed for use as a gelling agent for preparing a solid, rigid gel, it will likely comprise either a majority of kappa-carrageenan or a majority of a mixture of kappa and iota carrageenans. Pure kappa carrageenan for use herein (κ-carrageenan) is from Kappaphycus alverezii “cottonii,” and available from Danisco and other suppliers.

Kappa carrageenan from Kappaphycus alverezii “cottonii” is used in the present air freshener at an amount sufficient for gellation, which is preferably from about 0.1 wt. % to about 10 wt. %. The κ-carrageenan is most preferably present at about 1 wt. % to about 2 wt. %, based on the total weight of the aqueous air freshener gel. Commercial K-carry ageenan from Kappaphycus alverezii “cottonii” that finds use herein include, but are not limited to, Grindsted® brand products available from Danisco USA Inc., Danagel® brand products available from FMC, GenuGel® brand products available from CP Kelco, Carrageenan NF from AEP Colloids, Inc., AquaGel® brand products available from Marcel Trading Corporation, and the Eugel® brand products available from MCPI Corporation, particularly Eugel® AFG (“air freshener gel”).

Nonionic Cellulose Derivative

The present invention necessarily comprises a nonionic cellulose derivative. Addition of a very specific and unique nonionic cellulose derivative to a carrageenan gel composition unexpectedly and dramatically improves the physical stability of the final gel. In particular, the combination of methyl hydroxyethyl cellulose with κ-carrageenan dramatically reduces syneresis of the water-based gel and dramatically improves resistance to mottling over the course of repeated freeze-thaw cycling.

Useful nonionic cellulose derivatives include hydroxyethyl cellulose (HEC), ethylhydroxyethyl cellulose (EHEC), hydroxypropylhydroxyethyl cellulose (HPHEC), methyl cellulose (MC), methylhydroxypropyl cellulose (MHPC), methylhydroxyethyl cellulose (MHEC), hydrophobically modified hydroxyethyl cellulose (HMHEC), hydrophobically modified hydroxypropyl cellulose (HMHPC), hydrophobically modified ethylhydroxyethyl cellulose (HMFEHEC), hydrophobically modified hydroxypropylhydroxyethyl cellulose (HMHPHEC), hydrophobically modified methyl cellulose (HMMC), hydrophobically modified methylhydroxypropyl cellulose (HMMHPC), hydrophobically modified methylhydroxyethyl cellulose (HMMHEC), and mixtures thereof. These substances are available as Methocel®, Cellosize®; Amerchol; Natrosol®; Hercules and Blanose®; Aqualon, Aquasorb®, Ambergum®; Hercules; Cellgon®; Montello. More preferred are methyl hydroxyethyl cellulose (MHEC) and hydroxyethyl cellulose (HEC). Most preferred is to use methyl hydroxyethyl cellulose (MHEC). The nonionic cellulose derivative is incorporated into the water-based gel of the present invention at from about 0.5 wt. % to about 1.5 wt. % actives, based on the total weight of the composition.

Fragrance

The improved water-based gel of the present invention includes a fragrance. A fragrance in accordance with the present invention may comprise one of more volatile organic compounds available from any of the now known, or hereafter established, perfumery suppliers, such as International Flavors and Fragrances (IFF) of New Jersey, Givaudan of New Jersey, Firmenich of New Jersey, etc. Many types of fragrances can be used in the present invention. Preferably the fragrance materials are volatile essential oils. The fragrances, however, may be synthetically derived materials (aldehydes, ketones, esters, etc.), naturally derived oils, or mixtures thereof. Naturally derived fragrance substances include, but are not limited to, musk, civet, ambergis, castoreum and like animal perfumes; abies oil, ajowan oil, almond oil, ambrette seed absolute, angelic root oil, anise oil, basil oil, bay oil, benzoin resinoid, bergamot oil, birch oil, bois de rose oil, broom abs., cajeput oil, cananga oil, capsicum oil, caraway oil, cardamon oil, carrot seed oil, cassia oil, cedar leaf, cedarwood oil, celery seed oil, cinnamon bark oil, citronella oil, clary sage oil, clove oil, cognac oil, coriander oil, cubeb oil, cumin oil, camphor oil, dill oil, estragon oil, eucalyptus oil, fennel sweet oil, galbanum res., garlic oil, geranium oil, ginger oil, grapefruit oil, hop oil, hyacinth abs., jasmin abs., juniper berry oil, labdanum res., lavander oil, laurel leaf oil, lavender oil, lemon oil, lemongrass oil, lime oil, lovage oil, mace oil, mandarin oil, mimosa abs., myrrh abs., mustard oil, narcissus abs., neroli bigarade oil, nutmeg oil, oakmoss abs., olibanum res., onion oil, opoponax res., orange oil, orange flower oil, origanum, orris concrete, pepper oil, peppermint oil, peru balsam, petitgrain oil, pine needle oil, rose abs., rose oil, rosemary oil, sandalwood oil, sage oil, spearmint oil, styrax oil, thyme oil, tolu balsam, tonka beans abs., tuberose abs., turpentine oil, vanilla beans abs., vetiver oil, violet leaf abs., ylang ylang oil and like vegetable oils, etc. Synthetic fragrance materials include but are not limited to pinene, limonene and like hydrocarbons; 3,3,5-trimethylcyclohexanol, linalool, geraniol, nerol, citronellol, menthol, borneol, borneyl methoxy cyclohexanol, benzyl alcohol, anise alcohol, cinnamyl alcohol, f3-phenyl ethyl alcohol, cis-3-hexenol, terpineol and like alcohols; anethole, musk xylol, isoeugenol, methyl eugenol and like phenols; α-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, citral, citronellal, hydroxycitronellal, benzaldehyde, methyl nonyl acetaldehyde, cinnamic aldehyde, dodecanol, α-hyxylcinnamic aldehyde, undecenal, heliotropin, vanillin, ethyl vanillin and like aldehydes; methyl amyl ketone, methyl β-naphthyl ketone, methyl nonyl ketone, musk ketone, diacetyl, acetyl propionyl, acetyl butyryl, carvone, menthone, camphor, acetophenone, p-methyl acetophenone, ionone, methyl ionone and like ketones; amyl butyrolactone, diphenyl oxide, methyl phenyl glycidate, gamma.-nonyl lactone, coumarin, cineole, ethyl methyl phenyl glicydate and like lactones or oxides; methyl formate, isopropyl formate, linalyl formate, ethyl acetate, octyl acetate, methyl acetate, benzyl acetate, cinnamyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, methyl salicylate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, nopyl acetate, β-phenylethyl acetate, trichloromethylphenyl carbinyl acetate, terpinyl acetate, vetiveryl acetate and like esters, and the like. Suitable fragrance mixtures may produce an infinite number of overall fragrance type perceptions including but not limited to, fruity, musk, floral, herbaceous, edible, and woody, or perceptions that are in-between (fruity-floral for example). Typically these fragrance mixtures are compounded by the fragrance houses by mixing a variety of these active fragrance materials along with various solvents to adjust cost, evaporation rates, hedonics and intensity of perception. Well known in the fragrance industry is to dilute essential fragrance oil blends (natural and/or synthetic) with solvents such as ethanol, isopropanol, hydrocarbons, acetone, glycols, glycol ethers, water, and combinations thereof, to make the purchased fragrance raw material blend more easily handled by the formulator and to adjust the rate of evaporation of the volatiles and the hedonics. The preferred fragrance oil for use in the gel air freshener of the present invention may be comprised of a mixture of many fragrance actives and volatile solvents, sometimes along with smaller amounts of emulsifiers, stabilizers, wetting agents and preservatives. More often than not, the compositions of the fragrance oil purchased from the various fragrance supply houses remain proprietary and thus can only be described in general terms.

The fragrance material is preferably incorporated at a level of from about 0.001% to about 5% by weight in the water-based gel, based on the total weight of the finished composition. Typically the fragrance is added to a rapidly stirred and heated mixture of the carrageenan and water, in which it disperses.

Optional Ingredients

As mentioned above, water-based gels comprising carrageenan may also include additional ingredients to increase the stability of the solidified gel. Such materials include, but are not limited to: various natural gums derived from non-marine biological sources like gum Arabic, gum ghatti, gum tragacanth, Karaya gum, Guar gum, Locust Bean gum, beta-glucan, Chicle gum, Dammar gum, glucomannan, Mastic gum, Spruce gum, Tara gum, Cassia gum, Gellan gum, and xanthan gum, and mixtures thereof; and various small molecular weight salts like sodium chloride, potassium chloride, magnesium chloride, calcium chloride, sodium acetate, potassium acetate, magnesium acetate, calcium acetate, sodium sulfate, potassium sulfate, magnesium sulfate, and calcium sulfate. To further stabilize the water-based gel of the present invention, the total amount of these optional compounds in the finished gel may be from about 0.01 wt. % to about 10 wt. %, based on the total weight of the air freshener gel composition.

The water-based gel in accordance with the present invention may also include dyes, pigments or other suitable colorants to provide aesthetic appeal to the retail gel air freshener product. Such dyes may include FD&C and/or D&C Yellows, Reds, Blues, Greens and Violets, or really any other dye or pigment, and such raw materials are commonly purchased in either powder or liquid form from numerous suppliers. Dyes and/or pigments are incorporated at levels sufficient to provide light color to deep color to the solid gel product. When the optional dyes or pigments are incorporated to produce a colored air freshener gel, they are added at any time into the heated aqueous liquid gel mixture at from about 0.0001% to about 1% by weight, depending on the concentration of the colorants (e.g. if a liquid/diluted dye or a neat powder is the raw material). The water-soluble dyes will dissolve in the water whereas some pigments may only disperse.

The water-based gel in accordance with the present invention may also comprise one or more preservatives to help prevent dye fading and/or mold or other microbial growth in and/or on the gel. The preferred microbial preservatives include Neolone® and Kathon® products from Lonza and Rohm & Haas. These materials are incorporated at the manufacturers' recommended levels in the air freshener gel to discourage bacterial and mold growth. An ultraviolet inhibitor and/or an antioxidant such as BHT may also be added to the air freshener gel to reduce dye fading that may become an issue when the air freshener is opened and exposed to light by the consumer.

The water-based gel in accordance with the present invention necessarily includes water, and preferably the water is the ingredient present in the largest amount. For example, given the preferred wt. % ranges for the carrageenan gelling agent, the nonionic cellulose derivative, and the fragrance, the remaining wt. % of the composition is mostly water (even with optional adjuvant such as antimicrobials and colorants). Therefore it is preferred that the water-based gels of the present invention comprise at least 80 wt. % water, and more preferably at least 85 to 90 wt. % water.

Results and Discussion

TABLE 1 delineates an embodiment of the present invention, wherein a water-based gel comprises both κ-carrageenan and a nonionic cellulose derivative such as methyl hydroxyethyl cellulose. Gel A in TABLE 1 shows remarkable physical stability, with a measured syneresis of only 0.266, and very favorable freeze-thaw stability (no unaesthetic mottling appearance after repeated cycles).

TABLE 1
Gel air freshener

| | Gel A |
|---|---|
| Ingredients (wt. % actives) | |
| Kappa-carrageenan | 1-2 |
| Methyl hydroxyethyl cellulose | 0.5-1.5 |
| Hydroxyethyl cellulose | 0-0.4 |
| Calcium acetate | 0.1-0.7 |
| Potassium chloride | 0.05-0.50 |
| Fragrance | 1-5 |
| Preservative | Present |
| Water | q.s. |
| Total | 100 |
| Stability | |
| Syneresis | 0.266 |
| Freeze-Thaw cycling/resistance to mottled appearance | +++ |

TABLE 2 sets out the syneresis measured for eight water-based gels. Each gel composition contained 1.4 wt. % actives κ-carrageenan. The test gel then included an “additive” at the wt. % actives shown in the table.

TABLE 2
Screening of additional celluloses and gums

| | | wt. % | |
|---|---|---|---|
| Gel | Additive used in the gel composition | added | Syneresis |
| 1 | Methyl hydroxyethyl cellulose (inventive ref.) | 1.0 | 0.266 |
| 2 | Hydroxyethyl cellulose | 1.0 | 0.410 |
| 3 | Hydroxypropyl methyl cellulose | 1.0 | 0.564 |
| 4 | Carboxymethyl cellulose | 1.0 | 0.364 |
| 5 | Cassia gum | 1.0 | 0.610 |
| 6 | Iota-carrageenan | 1.0 | 0.190 |
| 7 | Locust bean gum (at 0.60 wt. %) | 0.6 | 1.110 |
| 8 | Hydroxypropyl guar | 1.0 | 0.510 |

As evident from TABLES 1 and 2, methyl hydroxyethyl cellulose uniquely improves the stability of a water-based carrageenan gel. More specifically, the combination of κ-carrageenan and methyl hydroxyethyl cellulose forms a remarkably stable gel that resists syneresis and mottled appearance even after repeated freeze-thaw cycles.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

We claim:

1. A stable water-based gel composition comprising: (a) from 0.001 wt. % to 5 wt. % fragrance; (b) from 0.1 wt. % to 10 wt. % κ-carrageenan from Kappaphycus alverezii “cottonii”; (c) from 0.5 wt. % to 1.5 wt. % nonionic cellulose derivative; and, (d) remainder water.

2. The composition of claim 1, wherein said nonionic cellulose derivative is methyl hydroxyethyl cellulose.

3. The composition of claim 2, further including hydroxyethyl cellulose.

4. The composition of claim 1, further including inorganic salts selected from the group consisting of potassium salts, and calcium salts, and mixtures thereof.

5. A method of increasing the freeze-thaw stability of a κ-carrageenan water-based gel, said method comprising the step of adding methyl hydroxyethyl cellulose to a water dispersion of κ-carrageenan and fragrance.

6. A use of methyl hydroxyethyl cellulose as a stabilizer for κ-carrageenan water-based gels.
